(19) United States
(12) Patent Application Publication
(10) Pub. No.: US 2011/0070597 A1
(43) Pub. Date: Mar. 24, 2011

(54) Thin-Film Passive Samplers for Detection of Hydrophobic Organic Contaminants and Estrogenicity in Various Environments

(75) Inventors: Penny Vlahos, Stonington, CT (US); Tom Harner, Toronto (CA)

(73) Assignee: UNIVERSITY OF CONNECTICUT, Farmington, CT (US)

(21) Appl. No.: 12/783,840

(22) Filed: May 20, 2010

Related U.S. Application Data
(60) Provisional application No. 61/216,696, filed on May 20, 2009.

(51) Int. Cl. G01N 33/00

(52) U.S. Cl. 435/7.8; 436/501; 422/68.1

(57) ABSTRACT

A thin-film passive sampler for use in detecting hydrophobic organic contaminants in air or aqueous environments. The sampler features a relatively thin layer of a suitable absorbent matrix coated directly on a solid support unit made of an inert material such as titanium or glass fibers. The passive samplers are simple and cost-effective to manufacture, easy to use, and exhibit rapid equilibration times and high accuracy.

### CROSS REFERENCE TO RELATED APPLICATION

This application claims the benefit of U.S. Provisional Application No. 61/216,696, filed May 20, 2009, the disclosure of which is incorporated by reference in its entirety.

### FIELD

This disclosure relates generally to a passive sampler for use in detecting the presence of hydrophobic organic contaminants (HOCs) and/or measuring heath effects, such as estrogenicity, of an environment. More specifically, the disclosure concerns rapid equilibrating passive samplers comprising a thin layer of a suitable absorbent matrix coated on an inert support structure. Embodiments of the disclosed samplers feature ethylene vinyl acetate (EVA) coated on a titanium plate, glass filter or like inert structure. Further embodiments comprise performance reference compounds and receptors embedded within the matrix layer.

### BACKGROUND

Measuring contaminants in marine systems is a challenge due to the nature of discreet sampling and the large volumes that typically need to be extracted. Currently, detection of organic contaminants in coastal systems is limited by such laborious techniques, resulting in limited chemical monitoring in observation networks. Passive sampling requires no external energy and allows the determination of accurate time weighted average or equilibrium concentrations. Passive sampling devices often mirror bioaccumulation rates in marine biota and can be a useful tool when determining toxicological effects of a local ecosystem. As passive samplers are fairly inexpensive, easy to use and small in size, they provide a cost effective alternative or complement to conventionally known grab sampling methods or large volume water extractions currently utilized for most long term water quality monitoring programs.

Pollution and contamination of shorelines, oceans and freshwater systems, in addition to air, is a growing global environmental concern, though monitoring of contaminants in all environments remains a challenge. Coastal observation networks have been introduced in several areas along the coastlines of the United States to provide continuous monitoring of environmental parameters (i.e., salinity, air/water temperature, wave height and wind speed and direction). Currently, the amount of chemical monitoring that can be conducted in these networks is restricted due to limitations in chemical sensor technology. There is a growing demand for improvements in detection capabilities in order to enhance monitoring efforts in these environments. Expanding observation networks to include chemical sensors (either active or passive samplers) for organic and/or other contaminants would provide important information to scientist, policy makers and the public.

Active sampling—the collection of discrete samples in the water phase—is the most common technique for monitoring water quality. A drawback of this approach is that episodic pollution events can easily be missed in environments where contaminant concentrations vary over time. Solutions to this problem include increasing sample frequency and/or using sampling systems that require a particular number of samples in any given period. Unfortunately, these solutions significantly increase experimentation time and expense, possibly prohibitively.

On the other hand, passive sampling is based on the free flow of an analyte from the sampled medium to a receiving phase in a sampling device. Passive samplers collect target compounds in situ without affecting the bulk solution. Based on a sampler's design, analysis of the sample may reflect either the concentration with which the device is at equilibrium or the time-averaged concentration to which the sampler was exposed. Passive samplers are relatively inexpensive, easy to deploy and require minimal monitoring while in situ. Consequently, passive samplers have the potential to become reliable and cost-effective tools for monitoring aquatic environments for the presence of pollutants.

Passive sampling devices vary greatly in size, shape, and material. Nearly all passive samplers share the same core design characteristics. First, passive samplers have a boundary layer between the sampled medium and the receiving phase. The diffusion across this boundary determines the rate at which the target analyte is sampled. When deployed, the target analyte accumulates in the sampler by diffusion through this static layer of water or by permeation through a membrane.

Secondly, it is preferable that a sampler be designed with a high A/V ratio (i.e., the ratio of surface area of the sampler to the volume of water sampled) to reduce equilibration times and ensure that low level concentrations of target compounds may be detected. The design of the sampler and the time left in situ determine the phase in which the sampler operates.

Many known passive sampling devices are large in size, complicated in design, difficult to transport, employ thick layers or units of receiving phase material (referred to herein as the “matrix”), and/or employ permeable or semi-permeable layers of material positioned between a receiving phase and testing environment. Devices with thicker receiving phase portions equilibrate at slower rates, and thus must be deployed for far longer periods of time prior to analysis.

### SUMMARY

There is a need for a rapid equilibrating passive sampler that is relatively small in size, is relatively simple to use and is easily and inexpensively manufactured.

### BRIEF DESCRIPTION OF THE DRAWINGS

FIG. 1A is an enlarged cross sectional view of a polymer coated glass sampler (POG) according to the disclosure;

FIG. 1B is an enlarged cross sectional view of an organic rapid equilibrating sampler (ORE) according to the disclosure;

FIG. 2 is a perspective view of a rapid equilibrating passive sampling device according to the disclosure;

FIG. 3 is a representative graph showing a general standard uptake curve of passive sampling devices;

FIG. 4 is a graph showing the uptake of BDE 17 in a disclosed POG as experimentally determined in a laboratory setting;

FIG. 5 is a graph showing the uptake of BDE 17 in a disclosed ORE as experimentally determined in a laboratory setting;

FIG. 6 is a graph depicting Log K_{EVA-W} versus Log K_{OW} plot determined for a disclosed POG; and

FIG. 7 is a graph depicting Log K_{EVA-W} versus Log K_{OW} plot determined for a disclosed ORE.

### DETAILED DESCRIPTION

An object of the disclosure is to provide a rapid equilibrating passive sampling device that is capable of sampling both air and aquatic systems to detect the presence of various organic contaminants both natural and anthropogenic under various environmental conditions. As used herein “anthropogenic” means derived or resulting from human activities, as opposed to those occurring in natural environments without human interference. As used herein to modify the layer of absorbent matrix, “thin” means a thickness of approximately 25 μm or less, preferably a thickness of 5 μm or less, and even more preferably a thickness of between 0.7 μm and 2 μm.

An embodiment of the device comprises a thin layer of a suitable matrix coated on a generally inert plate or similar structure.

One particular embodiment of the device features a relatively thing layer of ethylene vinyl acetate (EVA) coated on a thin titanium plate.

Another embodiment of the device features a relatively thin layer of EVA coated on a glass fiber filter.

In another embodiment of the disclosed rapid equilibrating passive sampler, a suitable matrix is embedded with receptor and coated on a generally inert plate or similar structure for measuring a targeted compound in the environment. For example, a suitable detector (for example, Estrogen Responsive Chemical Activated Luciferase Gene eXpression (ER-CALUX)) can be used to measure transactivation of the estrogen receptor in order to detect the presence of estrogenic substances. Molecules bound to the estrogen receptors will bioluminesce in the presence of such detectors. Users can thus screen the environment and gauge potential health risks rather quickly and easily.

Estradiol, a female sex hormone, has a direct effect on the function of the reproductive system, the nervous system, the cardiovascular system and the skeletal system. Blood sugar levels, skin and other tissues and functions are also significantly influenced by estradiol. Like all steroid hormones, excessive amounts of estradiol can contribute to a number of increased health risks. It is understood that many chemicals present in the environment mimic estrogen thereby disrupting normal endocrine function, and have raised public concern. It is believed that exposure to substances possessing estrogenic activity causes an increase in hormone-sensitive cancers, decreased sperm count in humans and compromised gonadal functionality in wildlife.

In a particular embodiment, the estrogen receptor luciferase is embedded within a layer of EVA and coated on either a titanium plate or glass fiber filter.

For clarity, the disclosure will describe the method and materials with respect to an aquatic environment, however the disclosed methods and materials are not limited to aquatic environments and this disclosure and claims encompass other environments, including air environments.

Further, the disclosed embodiments utilize ethylene vinyl acetate as the receiving matrix. However, any known chromatographic material can be substituted as appropriate or desired, depending on the types of compounds being monitored. These other materials can include, without limitation, organo-silicas, polymers and chelating and ion-exchange materials.

As used herein “polybrominated diphenyl ethers” belong to a group of brominated flame retardants (BFRs), which are the most commonly used, including hexabromcyclododecane (HBCD), tetrabromobisphenol A (TBBPA) and polybrominated biphenyls (PBBs). The disclosure presents experiments wherein levels of PBDEs present in various samples are measured via deployment of an embodiment of the disclosed passive sampler. However, the disclosed sampler can be used or adapted to measure virtually any known contaminant, including without limitation organic and inorganic compounds, such as pesticides, aromatic hydrocarbons, alkyl phenol compounds, halogenated aliphatic hydrocarbons, halogenated aromatic hydrocarbons, brominated diphenylethers, polychlorinated phenyls, chlorophenols, polycyclic aromatic hydrocarbons, phosphate triesters, phthalates, anionic detergents, organotin compounds, dioxins, furans and steroid hormones.

These compounds are used in plastics, textiles, electronic circuitry and other materials to reduce fire hazards by interfering with the combustion of the polymeric materials. Use of these compounds has increased since they were first introduced in the early 1960s.

Inclusion of brominated flame retardants (BFRs) are an inexpensive means of improving fire resistance in products, as compared to common alternatives such as phosphorus, metal based and inorganic compounds. BFRs can be utilized as either reactive or additive compounds. Reactive flame-retardants are incorporated in polymeric materials by covalent bonding between the polymer and the flame retardant. Additive flame-retardants are dissolved in the polymer. Additive flame retardants are often semi-volatile and may separate or leach from the surface of their product application over time gradually allowing the compounds to enter the environment. The potential for flame retardant release from additive retardants that are not chemically bonded is substantially greater than their covalently bonded reactive counterpart. PBDEs are incorporated into polymers, as additive flame retardants, in both commercial and domestic applications and are frequently added in concentrations of up to 30% by weight.

Brominated flame retardants are a diverse group of compounds; however, the flame retardant mechanism works similarly in all of the compounds. They decompose quicker than the polymeric matrix under heated conditions. This prevents the formation of flammable gases by chemical interference with the radical chain mechanism that occurs in the gas phase during combustion. The high energy H and OH radicals formed during combustion are scavenged by bromine radicals released from the additive flame retardant. PBDEs typically decompose approximately 50° C. below the polymer and therefore serve as effective flame-retardants. Aliphatic bromines are generally less thermally stable than aromatic compounds. Hence, aromatic bromine compounds are utilized the most widely. As such, these compounds have been found in measurable quantities over a broad geographical area in biological, water, and sediment samples.

Polybrominated diphenyl ethers are generally diphenyl ether molecules with between one and ten bromine atoms attached. Each individual PBDE variant, distinguished from others by the number of bromine atoms and the placement of the atoms, is referred to as a “congener” in the art. There are approximately 209 congeners of PBDEs in existence. Formula I shows the common PBDE structure wherein x+y=1-10.

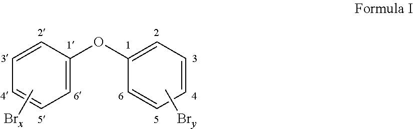

The 209 possible congeners are numbered according to the IUPAC system used for numbering polychlorinated biphenyls (PCBs) based on the position of the bromine atoms on the rings.

The disposal and recycling of products containing PBDEs release PBDEs into the environment. PBDEs remain intact in the environment for long periods, become widely distributed geographically, accumulate in the fatty tissue of living organisms and are toxic to humans and wildlife. Additionally, the lack of regulation on decaBDE usage coupled with the compound's suggested propensity for photodegradation to more toxic congeners suggest that secondary contamination will continue albeit in smaller quantities.

Environmental release of PBDEs may occur at various times throughout their lifetime. This includes initial synthesis, incorporation into polymers or related finished products, during use of products, or as a result of their disposal or recycling. The percentage of PBDEs in many commercially used polymers may be as high as 30% by weight and when used as an additive flame retardant. The leaching of PBDEs into the environment begins immediately during manufacture and continues through the entire life of the product and disposal or recycling.

Polybrominated diphenyl ethers are persistent organic pollutants ubiquitous in the environment. As such, they pose a threat to the health of wildlife and humans. High concentrations of BDE-47, -99, and -100 have been identified in biota (fish, birds, mammals) in aquatic and marine ecosystems. Estimates of environmental releases are difficult to obtain due to high variability and further research needs to be conducted to correctly quantify current emissions, sinks, and sources.

It would be desirable to have a simple, rapid-equilibrating passive sampling device from which levels of hydrophobic organic contaminants, such as PBDEs, in an environment—water or air—can be measured. Embodiments of the disclosed passive sampling devices were calibrated, deployed and used to determine the presence and levels of numerous PBDEs in a coastal water environment.

### Sampler Theory and Design

A passive sampling device collects chemical compounds without the provision of energy from an external source and is based on the free flow of an analyte from the sampled medium to a receiving phase in the sampling device. This net flow of analyte results from a gradient in the chemical potentials of the analyte across the separate phases. For environmental marine monitoring, the flow would occur from seawater to the polymer used in the passive sampler. Net movement continues until the sampling period is terminated or thermodynamic equilibrium is established. The exchange kinetics between sampler and water can be described by a first-order one-compartment model:

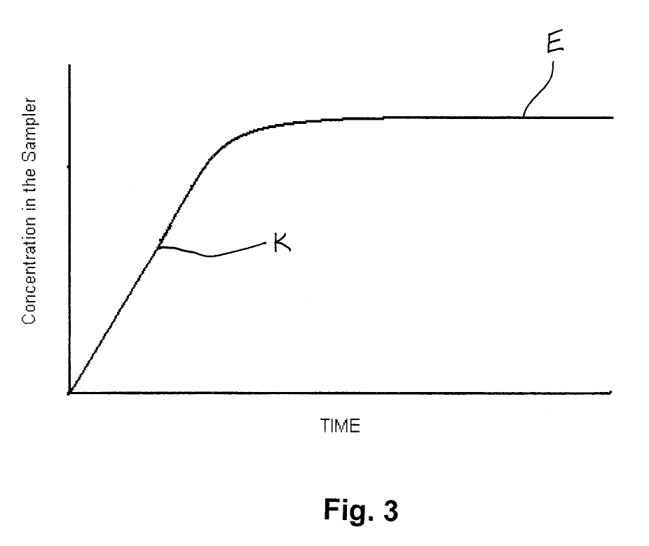

C_{S}(t)=C_{W}(k₁/k₂)(1−e^{-k}^{₂}^{t}) [Equation 1]

where C_{S}(t) is the contaminant concentration in the sampler polymer as a function of time, t, C_{W} is the contaminant concentration in the sampled medium (i.e., seawater), and k₁ and k₂ are the uptake and offload rate constants respectively. Passive samplers follow one of two phases in the accumulation of organic pollutants during field deployments. These phases are known as “kinetic” and “equilibrium”. The typical uptake of an analyte is illustrated in FIG. 3. As depicted in FIG. 3, the concentration in the sampler is linearly proportional to the duration of the deployment period in the kinetic phase (K). The concentration in the sampler remains steady once thermodynamic equilibrium has been reached (equilibrium phase; E).

### Kinetic Samplers

Kinetic devices sample under the assumption that the rate of mass transfer of the target analyte is linearly proportional to the difference in chemical potentials between the two partitioning phases. Kinetic samplers are used to determine time weighted average (TWA) concentrations of the target analyte in the sampled medium. In kinetic phase sampling, Equation 1 is reduced and rearranged to Equation 2:

Mₛ(t)=C_{w}Rₛt [Equation 2]

where Ms(t) is the mass of the analyte accumulated in the passive sampler at any time, t, and Rₛ is the sampling rate. The sampling rate is equivalent to the volume of water that would need to be extracted per unit time to account for the mass increase in the sampler.

Extensive calibration studies are often conducted at known exposure concentrations to characterize and parameterize this type of sampler and to assist in the accurate determination of TWA concentrations. Sampling rates may vary due to biofouling, turbulence, and temperature. Performance reference compounds (PRCs) may be used to help correct for deviations from standard environmental conditions. Compounds that do not occur in nature are used as PRCs and are placed in the sampler prior to deployment. Factors that influence the uptake of the target analyte will equally affect the offloading of the PRC. Utilization of these compounds allows for the in situ calibration of the sampler resulting in more accurate estimates of pollutant concentrations. The time weighted average water concentrations provided by these samplers may be valuable in assessing toxicological risks.

### Equilibrium Samplers

Equilibrium samplers remain in situ long enough to establish thermodynamic equilibrium between the water and the reference phase (receiving matrix). In this phase, Equation 1 reduces to Equation 3:

Cₛ=C_{w}K_{sampler,medium} [Equation 3]

Dissolved contaminant concentrations may be calculated with the knowledge of reference phase-water partition coefficients, K_{sampler,medium}. One of the challenges of sampling in the equilibrium phase is to reduce the time to reach equilibrium to a reasonable period. This is particularly important when using these devices in aquatic environments as biofouling can play a role in the degradation of sampler performance.

The ratio of the surface area of the sampler to the volume of water sampled (A/V) plays an important role in the rapidity of reaching equilibrium. Generally, a fast sampling device is characterized by a high A/V ratio. For thin-film sampling devices, the thickness of the film (polymer) will determine the rapidity with which the sampler reaches thermodynamic equilibrium—i.e., a thinner film sampler will equilibrate faster than a sampler with a thicker coating.

The time to reach equilibrium is a function of the partition coefficient K_{sampler,medium} and the uptake rate k₁ of the compound. Uptake follows a standard saturation curve in which the time to reach 90% of equilibrium can be expressed as:

t_{90%}=ln 10/k₂=K_{sampler,medium}(ln 10/k₁) [Equation 4]

### Basic Design

Passive sampling devices currently in use by the scientific community vary greatly in size, shape and material; however, nearly all passive samplers are based on the same design characteristics. First, passive samplers must have a barrier between the sampled medium and the receiving phase. The barrier determines the rate at which the target analyte is sampled. The properties of the barrier determine whether the sampler is classified as permeation-based or diffusion-based. The process of sampling the target analyte is the same in both types of devices. The target analyte(s) accumulate in the sampler either by permeation through a membrane or diffusion through a static layer of water.

Additionally, it is preferable to design a sampler with a high A/V ratio to reduce equilibration times and ensure that low level concentrations of target compounds may be detected. The design of the sampler and the time left in situ generally determine the phase in which the sampler operates.

### Calibration of a Polymer Coated Thin Film Passive Sampler

Extensive calibration studies are required to parameterize a passive sampler to correctly determine environmental concentrations based on sampler concentrations. These experiments must be conducted in the laboratory at known exposure concentrations and simulated environmental conditions to characterize the uptake of contaminants and to assist in the determination of equilibrium or time weighted average concentrations. These parameterizations may then be used to determine equilibration times, sampling rates, and partition coefficients. Accurate calculations of hydrophobic organic contaminants (HOC) water concentrations depend upon these experimentally determined values.

Passive sampling devices sense the target analyte in one of two phases: kinetic or equilibrium. When deployed as a kinetic sampler, the concentration in the sampler is linearly related to the length of the deployment period. In contrast, during the equilibrium phase, the concentration of the pollutant in the sampler remains constant once thermodynamic equilibrium has been reached (see FIG. 3).

The uptake phase determines the manner in which water concentrations are calculated. Experimental determination of the time to reach thermodynamic equilibrium ensures that the correct sampling time is used and that the sampler is not removed during the curvilinear portion of uptake, thereby providing accurate and predictable results for passive sampler derived water concentrations.

Water concentrations for samplers in the kinetic phase can be calculated using sampling rates. The sampling rate (RS) is equal to:

Rₛ=(D*A₍ₛₐₘₚₗₑᵣ₎)/L

or

Rₛ=Mₛ/(C_{w}*t) [Equations 5, 6]

where D is the diffusion coefficient of the compound of interest (m²d⁻¹), A₍ₛₐₘₚₗₑᵣ₎ is the area of the sampling device (m²), L is the diffusion distance of the compound (m), C_{W} is the water concentration (gL⁻¹), M_{S} is the mass of the compound detected in the sampler (g) and t is the time the sampler was in the water (days). The units for R_{S} are m³d⁻¹ and were converted to Ld⁻¹ for this study. It is known that experimentally determined sampling rates often deviate from those calculated from Equation 1 due to the non-ideal behavior of the target analyte. Therefore, experimental calibrations that simulate environmental conditions as closely as possible are the preferred method for determining sampling rates.

Accurate partition coefficients are necessary to derive water concentrations from the deployed sampler. At equilibrium and under ideal conditions, only the concentration within the sampler and the partition coefficient are needed to determine the analyte concentration in the medium. The partition coefficient for the sample can be established by analysis of the analyte in the medium and sampler once thermodynamic equilibrium is achieved. It is extremely important to constrain the parameters of the passive samplers and establish accurate partitioning coefficients. Several variables (e.g., salinity, temperature) in the environment may affect the partitioning coefficients and constraining these is necessary for proper application. For example, although estimates of octanolwater partition coefficients for PBDE congeners are reasonably established, reported values can vary by an order of magnitude.

The polymer coated glass samplers (POG) and the organic rapid equilibrating (ORE) samplers, both comprising a thin film (generally ˜1-2 μm) of ethylene vinyl acetate (EVA), were calibrated by experimental determination of EVA-Water partition coefficients and sampling rates. The ORE is comprised of a 1.4 μm EVA coating on a 5.1×7.6 cm titanium plate, while the POG utilizes a thin-film of EVA on a glass fiber filter. A closed system experiment was used to calibrate the POG and ORE devices in this study. Sampling rates and equilibration times were determined for fourteen PBDE congeners. Ethylene Vinyl Acetate (EVA)—Water partitioning coefficients (K_{EVA-W}) were experimentally determined for 5 BDE congeners. A Log K_{EVA-W}−Log K_{OW} (Octanol-Water partitioning coefficient) plot was constructed and used to define Log K_{EVA-W} partitioning coefficients for the 14 BDE congeners examined (values ranged from 6.39 to 10.6). The experimental design was successful in parameterizing the desired constants.

### Experimental 1

### Preparation of POG Sampler

A coating solution of EVA (Elvax 40W, DuPont) was created by dissolving EVA pellets in dichloromethane (DCM), followed by systematic dipping of a glass fiber filter directly into the EVA/DCM solution and evaporation of the DCM. Average mass of the EVA was 0.04 g±0.003 g per POG (density EVA=0.93 gmL⁻¹). After weighing, the samplers were attached to a stainless steel frame, wrapped in pre-cleaned aluminum foil, placed in an airtight container and stored at −4° C. until deployment.

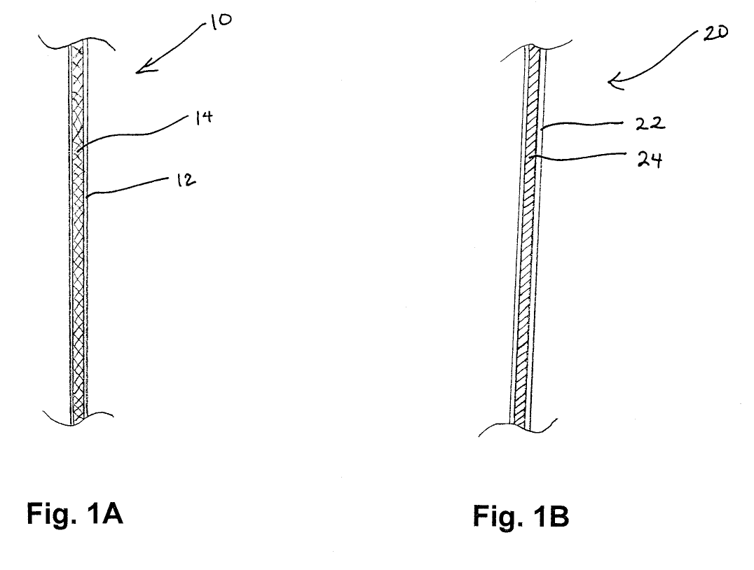

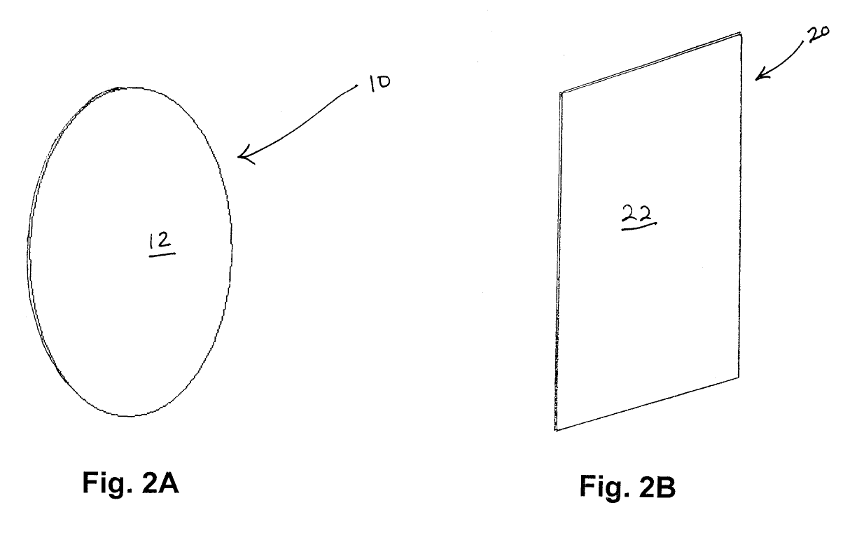

FIG. 1A depicts an enlarged partial cross sectional view of a POG sampler 10 prepared by the process described in Experimental 1. As can be seen, a thin layer of EVA matrix 12 is coated onto a glass fiber filter 14. When deployed, the matrix 12 will be directly exposed to the testing environment. FIG. 2A is a perspective view of the POG sampler 10. The matrix 12 is preferably ˜1-2 μm thick.

It should be noted that the particular size and shape of the described POG is non-limiting. Also, while the POG sampler 10 features an EVA coating 12, EVA can be substituted by a different suitable matrix, as desired. Further, the particular described process of dipping the glass filter 14 is non-limiting. Other coating processes can be employed to prepare the POG sampler 10, such as for example, spray coating.

### Experimental 2

### Preparation of ORE Sampler

The same coating solution used for the POGs in Experimental 1 was utilized for ORE preparation. Titanium plates were dipped into the solution and dried, then re-dipped and re-dried, leaving an approximately 1.4 μm (as determined by EVA mass and ORE surface area) coating of EVA on the titanium plate, and thus creating an ORE with an approximate surface area of over 77.5 cm². The average mass of the EVA was 0.01 g+/−0.001 g per ORE (density EVA=0.93 gmL⁻¹). After weighing, the samplers were attached to a stainless steel frame, wrapped in pre-cleaned aluminum foil, placed in an airtight container and stored at −4° C. until deployment.

With reference to FIG. 1B, an ORE sampler 20 prepared by the method of Experimental 2 is shown in enlarged partial cross sectional view. A thin layer of EVA matrix 22 is coated onto a titanium plate 24. The matrix 22 is preferably ˜1-2 μm thick. FIG. 2 is a perspective view of the ORE sampler 20.

Similar to the POG sampler 10, the ORE sampler 20 is not limited in size or shape, nor is the preparation process or specific matrix (EVA) limiting.

### Experimental 3

### Calibration of POG and ORE Samplers

The polymer coated glass samplers (POG) and the organic rapid equilibrating (ORE) samplers, 10 and 20, both consisting of a thin film (on the order of ˜1-2 μm) of EVA, were calibrated by experimental determination of EVA-Water partition coefficients and sampling rates. The ORE is comprised of a 1.4 μm EVA coating on a 5.1×7.6 cm titanium plate, while the POG utilizes a thin-film of EVA on a glass fiber filter.

A closed system experiment was used to calibrate the POG and ORE devices. Sampling rates and equilibration times were determined for fourteen PBDE congeners. EVA-Water partitioning coefficients (K_{EVA-W}) were experimentally determined for 5 BDE congeners. A Log K_{EVA-W}−Log K_{OW} (Octanol-Water partitioning coefficient) plot was constructed and used to define Log K_{EVA-W} partitioning coefficients for the 14 BDE congeners examined. Values ranged from 6.39 to 10.6. The experimental design was successful in parameterizing the desired constants. In sum, both samplers were successfully calibrated.

### Experimental 4

### Tank Deployment of Samplers

A dichloromethane rinsed 20 gallon fish tank was employed for the tank deployment experimental. Three POGs and three OREs were suspended from a stainless steel frame proximate the top of the tank.

Once dry, the tank was filled with artificial seawater comprising sodium chloride, sodium bicarbonate, magnesium sulfate, and high purity Milli-Q water. A handheld multiparameter instrument (YSI-556) was used to confirm salinity (31 parts per thousand), dissolved oxygen (0.19 mM), and temperature (20° C.) prior to starting the experiment. The tank was fit with a plexiglass lid sealed with polytetrafluoroethylene (PTFE; i.e., Teflon®) and duct tape.

The tank was spiked with 0.2 mL of the PBDE Predominant Congener Mixture in n-nonane from Cambridge Isotope laboratories and allowed to equilibrate for 48 hours. Samplers were removed from refrigeration and allowed to equilibrate to room temperature. 400 mL of water was taken to establish the initial concentration. Seven sets of three samplers were added to the tank. The tank was secured with a lid, PTFE and duct tape, as described above. Four hundred milliliter aliquots of tank water and one sampler set were removed at the following time intervals: 1 hr, 2 hrs, 6 hrs, 12 hrs, 3 days, 12 days, and 32 days. The tank was open to the air in the fume hood for less than 4 minutes during each sampling period. After removal, each sampler set was wrapped in pre-cleaned aluminum foil and placed frozen in an airtight bag at −20° C. until extraction and analysis. Water-side 400 mL aliquots were frozen at −20° C. until extraction and analysis.

The POGs and OREs were extracted for analysis of PBDE concentration.

Extracts were analyzed for BDE congeners 17, 28, 47, 66, 71, 85, 99, 100, 138, 153, 154, 183, 190, and 209 by gas chromatography-mass spectrometry (GC/MS). Masses 79 (PBDEs) and 414 (Mirex) were used for quantification with Masses 81 (PBDEs), 487, 486 (DecaBDE), and 412 (Mirex) acting as confirmation ions. Extracts were identified and quantified by comparison with known concentrations of prepared standard solutions.

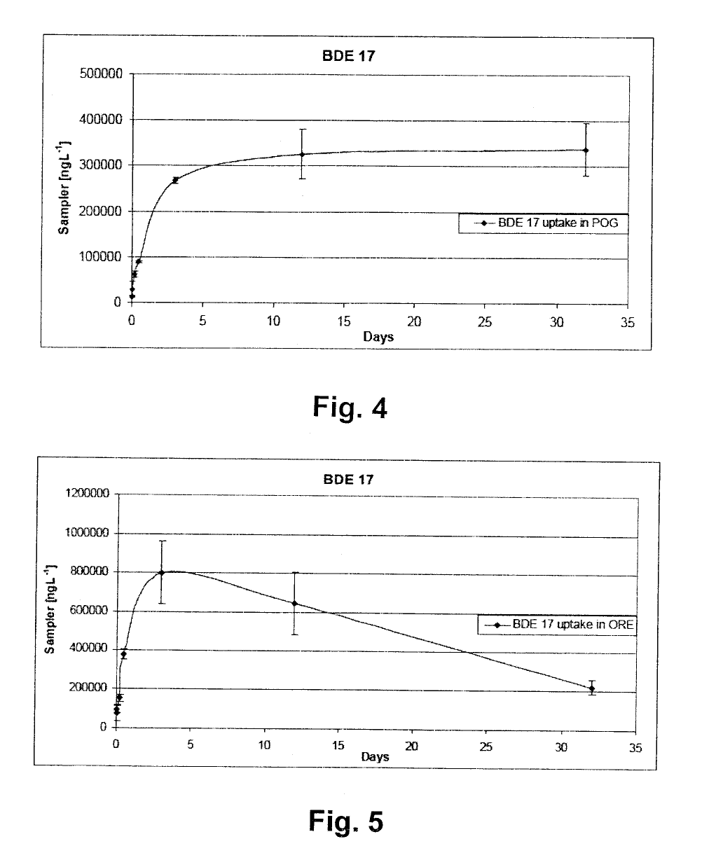

Uptake curves for BDE 17 in the POG and ORE are shown in FIGS. 4 and 5, respectively. As indicated, the POGs reached equilibrium in 12 to 32 days (FIG. 4), while the OREs equilibrated in 3 to 12 days and then offloaded as water concentrations decreased (FIG. 5). The disparity between equilibration times is likely due to the non-uniformly coated nature of the POG versus the more uniformly coated ORE. Notably, while the equilibration times differ, they do not exhibit large variability. Consequently, it can be assumed that the majority of the glass filter has an EVA coating thickness that is similar to the titanium plate.

Table 1 shows the sampling phase for the ORE and POG at the end of 14 days of deployment.

TABLE 1

| BDE | ORE | POG |
|---|---|---|
| 17 | Equilibrium | Equilibrium |
| 28 | Equilibrium | Equilibrium |
| 47 | Equilibrium | Equilibrium |
| 66 | Equilibrium | Equilibrium |
| 71 | Equilibrium | Equilibrium |
| 85 | Equilibrium | Kinetic |
| 99 | Equilibrium | Kinetic |
| 100 | Equilibrium | Kinetic |
| 138 | Equilibrium | Kinetic |
| 153 | Equilibrium | Kinetic |
| 154 | Equilibrium | Kinetic |
| 183 | Kinetic | Kinetic |
| 190 | Kinetic | Kinetic |
| 209 | Kinetic | Kinetic |

Sampling rates were calculated experimentally using Equation 2. The sampling rates varied between sampler types. The differences in sampling rates were likely due to variations in the thickness and heterogeneity versus homogeneity of the thin film coating on the respective plates. Sampling rates (R_{S}(s)) were calculated at 1, 2, or 6 hrs depending upon the shape of the uptake curve. Table 2 summarizes the experimentally determined R_{S}(s) values for the POG and ORE.

TABLE 2

| BDE | Rs (Ld⁻¹) ORE | Rs (Ld⁻¹) POG |
|---|---|---|
| 17 | 6.4 | 3.2 |
| 28 | 7.6 | 4.8 |
| 47 | 4.9 | 9.9 |
| 66 | 5.6 | 6.9 |
| 71 | 6.1 | 7.6 |
| 85 | 8.8 | 8.2 |
| 99 | 4.0 | 6.5 |
| 100 | 4.2 | 4.5 |
| 138 | 4.1 | 5.1 |
| 153 | 2.8 | 6.5 |
| 154 | 2.6 | 5.7 |
| 183 | 8.6 | 7.5 |
| 190 | 10.4 | 11.4 |
| 209 | 7.5 | 6.7 |

Partitioning coefficients were calculated for the five unsaturated BDE congeners at equilibrium and are summarized in Table 3:

TABLE 3

LOG K_{EVA-W}

| BDE | POG | ORE |
|---|---|---|
| 17 | 6.01 ± 0.07 | 6.40 ± 0.08 |
| 28 | 5.90 ± 0.06 | 6.38 ± 0.10 |
| 47 | 6.43 ± 0.07 | 6.79 ± 0.10 |
| 66 | 6.25 ± 0.07 | 6.66 ± 0.11 |
| 71 | 6.24 ± 0.05 | 6.67 ± 0.02 |

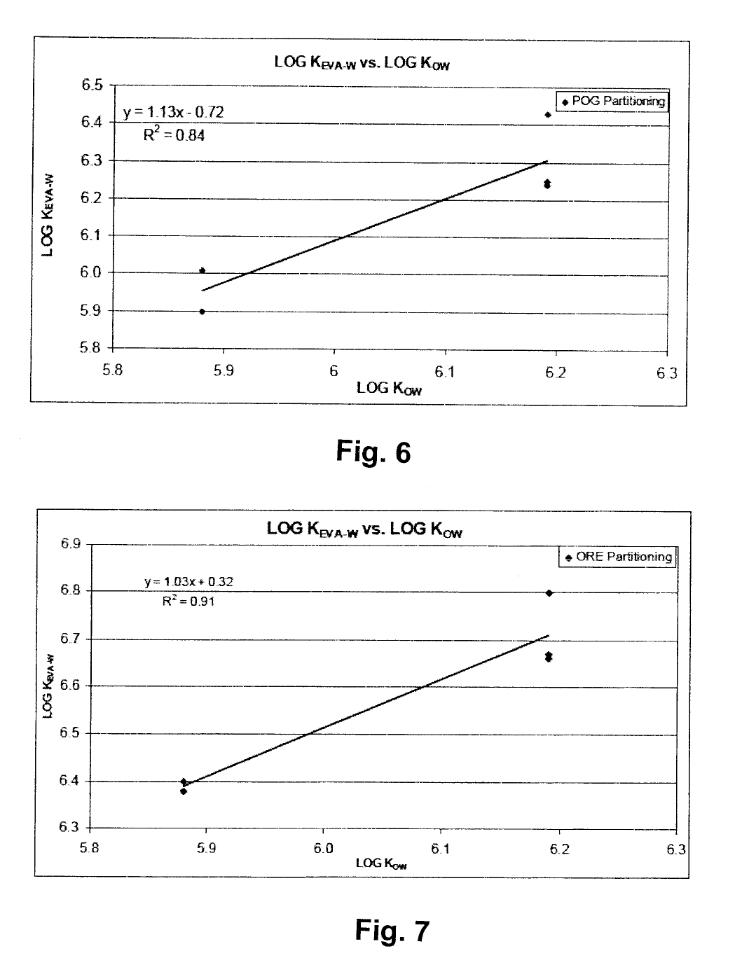

FIGS. 6 and 7 show plots of Log K_{EVA-W} versus Log K_{OW} values for these five congeners for the POG and ORE, respectively.

As can be seen, the partitioning experiment was successfully completed with sampling rates, equilibration times, and partitioning coefficients determinations made for each type of sampler. The degree of saturation for each congener appeared to affect the uptake of individual congeners. Log KEVA-W for the PBDE congeners ranged from 6.39 to 10.6 and sampling rates ranged from 3.2 to 11.4 Ld⁻¹. The data collected using the disclosed thin film samplers is supported by previous studies on halogenated pesticides.

### Experimental 5

### Deployment of POG in Coastal Waters

Polymer-coated glass samplers (POGs) were deployed in the Thames River Estuary, located in southeastern Connecticut, to test their effectiveness in aquatic systems when used to identify, quantify and assess the distribution of HOCs. The samples were analyzed for concentrations of polybrominated diphenyl ethers (PBDEs) and currently used pesticides (CUPs).

The POG sampler used in this study comprises a 12.5 cm diameter glass fiber filter coated with a thin layer of EVA. The sampler was placed within a stainless steel cage and deployed.

Samplers were deployed in situ for 12 days (long enough to establish thermodynamic equilibrium for all target analytes except BDEs 99 and higher, which were still in the kinetic phase). In the equilibrium phase, water concentrations (C_{W}) may be calculated using the following equation:

C_{W}=C_{S}/K_{EVA-W} [Equation 7]

where K_{EVA-W} is the partition coefficient of the target analyte for EVA and water. For passive samplers in the kinetic phase, Equation 2 may be used to calculate the requisite water concentration:

C_{W}=M_{S}(t)/(Rₛ*t) [Equation 8]

where M_{S}(t) is the mass of the target analyte accumulated in the passive sampler and R_{S} is the sampling rate. Dissolved contaminant concentrations in EVA may be calculated with the knowledge of reference phase-water partition coefficients. A detailed description of equilibrium partitioning for organic compounds is provided in Schwarzenbach R., et al., 2003. Environmental Organic Chemistry, Second Edition. John Wiley & Sons, Inc., New York.

### Design

In total, three EVA coated POG samplers and three generally equivalent field blanks were prepared. Samplers were deployed at three stations along the estuary. Each station was located adjacent to potential sources of the targeted organic contaminants such as a chemical manufacturing plant, U.S. Naval Submarine Base, and pharmaceutical company.

The POG was suspended on a stainless steel frame approximately 50 cm above the bottom sediments and left in situ for 12 days. Field blanks were exposed on deck until the respective POG entered the water. The blanks were then wrapped in pre-cleaned foil, placed in an airtight container and stored at −4° C. until ready for extraction and analysis.

The POGs were recovered at the end of the 12 day time period. Conductivity, temperature, and depth (CTD) profiles were recorded at each station using a Seabird CTD. Bottom water salinity and temperature ranges varied only slightly between stations. Salinity readings were 30 parts per thousand (ppt) at Station 1 and 31 ppt at Stations 2 and 3. Bottom water temperatures ranged from 18-20° C.

Samplers were extracted as described herein after warming to room temperature and being rinsed with de-ionized water to remove algal debris and sediment from the stainless steel cage.

### Analysis

Extracts were analyzed for BDE congeners 17, 28, 71, 47, 66, 100, 99, 85, 154, 153, 138, 183, 190, and 209 by gas chromatography-mass spectrometry (GC/MS). Extracts were identified and quantified by comparison with known concentrations prepared in standard solutions. Currently Used Pesticides were analyzed via gas chromatography/mass spectrometry as described by Yao Y., et al., 2006, Spatial and temporal distribution of pesticide air concentrations in Canadian agricultural regions. Atmos. Environ. 40 (23), 4339-4351, using both electron impact ionization (EI) and negative chemical ionization (NCI) modes.

EI mode was utilized in the analysis of the following pesticides: phorate, dazomet, simazine, carbofuran, atrazine, terbufos, diazinon-d10, diazinon, disulfoton, alachlor, metolachlor, and parathion-d10. NCI mode was used for analysis of the following: metribuzin, malathion, parathion-d10, chlorpyrifos, dacthal, pendimethalin, α-endosulfan, diazinon-d10, hlorothalinol, trifluralin, α-HCH-d6, and α-HCH.

Water concentrations were derived using Equation 7 for the contaminants in the equilibrium phase. These contaminants include the pesticides and BDEs 17, 28, 47, 66 and 71. Equation 8 was utilized for the conversion of GC/MS data to water concentrations for BDEs 85 and higher. Water concentrations were successfully calculated for all subject contaminants.

Of the nineteen currently-used pesticides that were targeted, six were detected. Experimentally determined blank-corrected water concentrations for the detected pesticides at the three subject stations in the estuary are shown in Table 4:

TABLE 4

| Target Analyte | Station 1 | Station 2 | Station 3 |
|---|---|---|---|
| Atrazine (ng/L) | 47 | 74 | 72 |
| Metribuzin (ng/L) | 110 | 110 | 110 |
| Chlorothalonil (ng/L) | 6.8 | 6.4 | 6.3 |
| Pendimethalin (pg/L) | 38 | 33 | 34 |
| A-Endosulfan (pg/L) | 120 | 63 | 64 |
| Trifluralin (pg/L) | 1.4 | 0.5 | 0.8 |

Experimentally determined blank-corrected water concentrations for subject PBDEs at the three subject stations in the estuary are shown in Table 5. Note that PBDE concentrations at Station 2 fell below detection limits.

TABLE 5

| Target Analyte (pg/L) | Station 1 | Station 2 | Station 3 |
|---|---|---|---|
| BDE 17 | 1.2 | <DL | 0.23 |
| BDE 28 | 0.56 | <DL | 0.56 |
| BDE 47 | 18 | <DL | 7.7 |
| BDE 66 | 0.38 | <DL | 0.30 |
| BDE 71 | 0.55 | <DL | 0.14 |
| BDE 85 | 0.76 | <DL | 0.49 |
| BDE 99 | 39 | <DL | 23 |
| BDE 100 | 17 | <DL | 9.7 |
| BDE 153 | 0.40 | <DL | 0.39 |
| BDE 154 | 0.94 | <DL | 0.31 |

### Experimental 6

### Detection of PBDEs by POGs and OREs in Bottom Waters

Glass fiber filter and titanium plate preparations were conducted in accordance with the disclosed methods. POGs and OREs were prepared for both deployment and as blanks. The POGs (n=3 per station) and OREs (n=4 per station) were suspended approximately 50 cm above the bottom sediments and left in situ for 14 days. During sampler deployment, conductivity, temperature, and depth (CTD) profiles were recorded at each station.

Recovery and extraction of the POGs and OREs was conducted as described above.

Extracts were analyzed for BDE congeners 17, 28, 47, 66, 71, 85, 99, 100, 138, 153, 154, 183, 190, and 209 by gas chromatography-mass spectrometry (GC/MS). Extracts were identified and quantified by comparison with known concentrations prepared in standard solutions and water concentrations were calculated. A summary of the blank corrected water concentrations by sampler type and station is found in Table 6:

TABLE 6

| | Station 1 | | Station 2 | | Station 3 | |
|---|---|---|---|---|---|---|
| BDE | POG | ORE | POG | ORE | POG | ORE |
| 17 | ND | 3.99 | <DL | 8.26 | 0.60 | ND |
| 28 | 0.73 | 7.22 | 1.57 | 4.45 | <DL | 0.93 |
| 47 | <DL | 50.71 | 1.49 | <DL | <DL | <DL |
| 66 | 0.58 | ND | 0.67 | <DL | 0.30 | ND |
| 71 | ND | ND | 0.40 | ND | 0.13 | ND |
| 85 | 0.33 | 0.51 | 2.25 | 0.74 | 0.35 | 0.15 |
| 99 | <DL | <DL | 7.86 | <DL | <DL | <DL |
| 100 | 2.62 | 1.88 | 3.68 | 0.28 | 1.20 | 0.05 |
| 153 | 2.03 | 0.38 | 0.96 | 0.15 | 0.37 | ND |
| 154 | 0.14 | 0.17 | 1.74 | ND | 0.07 | ND |
| 183 | 1.59 | 7.03 | 1.35 | 1.01 | 0.45 | 19.97 |
| 209 | 28.16 | 38.50 | 30.89 | ND | 1.45 | ND |

(unit = pg/L; ND = not detected; <DL = below detection limit)

As can be seen, POGs and OREs were successfully used to detect HOC concentrations in the bottom waters of the Thames River Estuary. Both EVA-coated substrates were effective in collecting the target analytes it does not appear that bromine substitution patterns preferred one substrate over another.

As illustrated in the preceding Experimentals, the disclosed POGs and OREs are simple, effective and efficient instruments for successfully detecting HOC concentrations in prepared water samples and in a coastal aquatic environment.

The ease of use and spatial resolution that POGs allow make them particularly useful for measuring average ambient concentrations and for identifying potential point sources. Additionally, they are less sensitive to extreme variations in pollutant concentrations in natural water systems and may be customized to provide uptake concentrations for variable term periods of environmental conditions, rather than single snapshots. Thickness of a matrix has a significant effect on equilibrium speed. Thus, the relatively thin layer employed in the disclosed samplers, as compared to known passive samplers, allow the disclosed samplers to reach equilibrium more efficiently.

What is claimed is:

1. A rapid equilibrating passive sampler for detecting the presence of contaminants in an environment, comprising:
an inert solid support unit; and
at least one layer of an absorbent matrix coated directly on said support unit.

2. The passive sampler of claim 1, wherein said absorbent matrix is ethylene vinyl acetate.

3. The passive sampler of claim 1, wherein said solid support unit is a titanium plate.

4. The passive sampler of claim 1, wherein said solid support unit is a glass fiber filter.

5. The passive sampler of claim 1, wherein said at least one layer of absorbent matrix has a thickness of between approximately 0.5 micrometers and approximately 3.0 micrometers.

6. The passive sampler of claim 1, wherein said at least one layer of absorbent matrix has a thickness of between approximately 1.0 and 2.0 micrometers.

7. The passive sampler of claim 1, wherein said rapid equilibrating passive sampler is prepared by the process of:
providing said inert solid support unit;
preparing a solution comprising an absorbent matrix compound dissolved in a solvent;
dipping said support unit at least once into said solution to yield a solution-enveloped support unit;
removing said solution-enveloped support unit from said solution;
evaporating said solvent.

8. The passive sampler of claim 7, wherein said absorbent matrix compound is ethylene vinyl acetate and said inert solid support unit is chosen from the group consisting of a glass fiber filter and a titanium plate.

9. The passive sampler of claim 1, comprising an estradiol receptor embedded within said absorbent matrix.

10. The passive sampler of claim 1 configured for detecting contaminants in an aqueous environment via deploying therein for a predetermined duration.

11. The passive sampler of claim 10 configured for detection of contaminants that are anthropogenic and/or naturally occurring aromatic brominated compounds and/or pesticides.

12. The passive sampler of claim 1 configured for detecting contaminants in an air environment via deploying therein for a predetermined duration.

13. A method of quantitating target contaminants in an environment, comprising:
preparing a passive sampler comprising an inert solid support unit coated with a thin layer of an absorbent matrix;
calibrating said passive sampler for said target contaminants;
deploying said passive sampler in said environment for a predetermined period of time;
extracting absorbed target contaminants from said absorbent matrix; and
calculating the concentration of said absorbed target contaminants in said environment.

14. The method of claim 13, wherein said step of calibrating includes determining the partition coefficient of the sampler for each of the target contaminants.

15. The method of claim 13, wherein the environment is an aqueous environment.

16. The method of claim 13, wherein said sampler is configured to detect the level of estrogenicity in said environment, comprising the additional step of embedding an estradiol receptor within said absorbent matrix.

17. The method of claim 16, comprising the additional steps of exposing said estradiol receptor to Estrogen Responsive Chemical Activated Luciferase Gene eXpression (ER-CALUX) and detecting bioluminescence of said estradiol receptor.

18. A rapid equilibrating passive sampler for detecting estrogenicity of an environment, comprising:
an inert solid support unit; and
an estrogen receptor embedded within an absorbent matrix, wherein
said matrix embedded with said estrogen receptor is coated on said solid support unit.

19. The passive sampler of claim 18, wherein the solid support unit is chosen from a group consisting of a titanium plate and a glass fiber filter.

20. The passive sampler of claim 18, wherein the estrogen receptor is an estradiol receptor.
